(19) United States
(12) Patent Application Publication
(10) Pub. No.: US 2020/0289734 A1
(43) Pub. Date: Sep. 17, 2020

(54) Sorbent Processing of Dialysate to Increase Solute Removal During Hemodialysis

(71) Applicant: The Board of Trustees of the Leland Stanford Junior University, Stanford, CA (US)

(72) Inventor: Timothy W. Meyer, Palo Alto, CA (US)

(21) Appl. No.: 16/795,954

(22) Filed: Feb. 20, 2020

Related U.S. Application Data
(60) Provisional application No. 62/816,632, filed on Mar. 11, 2019.

(51) Int. Cl. A61M 1/16; A61M 1/34; C01B 32/30

(52) U.S. Cl. CPC A61M 1/1696; A61M 1/3406; A61M 1/1605; A61M 2202/0498; A61M 1/1654; C01B 32/30; A61M 1/3465

(57) ABSTRACT

A hemodialysis device and method are provided in which a hemodialyzer is designed for blood to flow past a semipermeable membrane and for a dialysate to flow in the opposite direction on the other side of the semipermeable membrane where uremic solutes are capable of diffusing from the blood into the dialysate passing the semipermeable membrane, and further designed in which a sorbent incorporated in a dialysate path as the dialysate passes the semipermeable membrane. The sorbent binds to non-urea uremic solutes and in particular to non-urea uremic solutes that bind to plasma proteins. In addition, the sorbent binds urea and inorganic ions much less effectively than non-urea uremic solutes and in particular to non-urea uremic solutes that bind to plasma proteins.

### CROSS-REFERENCE TO RELATED APPLICATIONS

This application claims priority from U.S. Provisional Patent Application 62/816632 filed Mar. 11, 2019, which is incorporated herein by reference.

### FIELD OF THE INVENTION

The present invention relates generally to hemodialysis systems. More particularly, the invention relates to a hemodialysis system with a sorbent in the dialysate path as it passes the dialysis membrane.

### BACKGROUND OF THE INVENTION

Approximately 500,000 people are maintained on dialysis in the United States today. Most of them receive hemodialysis. Current treatment keeps patients alive but not well while consuming more than 7 percent of the Medicare budget. Worldwide, the number of patients on maintained on dialysis is greater than 2,000,000 and growing rapidly.

In hemodialysis, as illustrated in FIG. 1, blood 110 is pumped passed a stream of clean salt water, called dialysate 120, which flows in the opposite direction to the blood stream. The blood is separated from the dialysate by a semipermeable membrane 130, which is permeable only to small molecules. Small uremic solutes that have accumulated in the body diffuse into the dialysate and are carried down the drain. The majority of hemodialysis patients receive treatment 3 times per week for 3-4 hours each time.

Current hemodialysis treatment provides a high clearance of urea and other small uremic solutes that do not bind to plasma proteins. The clearance of protein-bound solutes is however poor. Effective plasma levels of these solutes may be more than 100-fold normal as compared to an average of about 5-fold above normal for urea. This is a serious defect as protein-bound solutes may be among the most toxic of uremic solutes. Indeed protein-binding normally provides a means to keep effective levels of toxic solutes very low in the body until they reach the kidney for removal.

Sorbents have been used to remove uremic solutes from the body, however their use has had very limited success and they are not part of current practice. The following are some examples.

Direct Sorbent Processing of Blood for Blood Plasma

Initial attempts to pass blood directly over sorbents failed because the sorbents interacted with platelets and complement. Later devices, in which sorbent particles were encapsulated, were ineffective because of encapsulation limited diffusion of solutes from plasma into the sorbent. A design in which plasma was separated from blood cells before passing over sorbents failed because it caused clotting. Bench testing showed that blood can be passed directly over sorbents to remove low molecular weight proteins but not the smaller molecules which constitute the majority of uremic solutes.

Sorbent Regeneration of Dialysate for Hemodialysis

In an early system the full dialysate stream was passed through a sorbent cartridge in which different successive elements removed urea, inorganic ions, and other uremic solutes. This system was designed to regenerate dialysate after each passage over the dialysis membrane and allow dialysis where the water supply was limited. Dialysate would pass by membrane, be regenerated in the sorbent cartridge, and then pass the membrane again, so that dialysis could be performed with only a few liters of dialysate instead of standard volumes of more than 100 liters. The combination of multiple sorbent layers to remove urea, inorganic ions, and other uremic solutes was costly and performed imperfectly. Though used for a while in water-poor areas, it was discontinued as systems for generating clean water improved. An attempt about ten years ago to revive full-stream dialysate regeneration with a modified multiple element sorbent cartridge did not lead to its successful re-introduction into practice. Sorbent regeneration of dialysate would also be a necessary feature of designs for wearable dialysis devices. However, the sorbent designs proposed so far are similar to the multiple-element cartridge originally used to regenerate dialysate in water-poor areas. They would be very costly and are far from proving practicable.

Addition of a Sorbent to the Dialysate

The inventor of this application has shown that adding a finely powdered sorbent to the dialysate would increase the clearance of protein-bound uremic solutes relative to urea without increasing the amount of dialysate required. Addition of a powdered sorbent to the dialysate reduces the free level of bound solutes in the dialysate as the dialysate passes by the membrane and thereby increases the rate at which solutes pass through the membrane from the blood into the dialysate. While addition of a finely powdered sorbent to the dialysate worked in bench testing, it was considered impractical for clinical use. Readily apparent disadvantages included the requirement for new dialysate systems capable of adding a sorbent to the dialysate throughout the course of treatment, maintaining the sorbent in suspension in the dialysate, and pumping the sorbent containing dialysate at accurate flow rates allowing volume removal from patients by ultrafiltration.

The present invention is an attempt to overcome at least some of the problems or issues in the art which currently provides poor clearance of uremic solutes that bind to plasma proteins.

### SUMMARY OF THE INVENTION

A hemodialysis device and method are provided in which a hemodialyzer is designed for blood to flow past a semipermeable membrane and for a dialysate to flow in the opposite direction on the other side of the semipermeable membrane where uremic solutes are capable of diffusing from the blood into the dialysate passing the semipermeable membrane, and further designed in which a sorbent (e.g. activated carbon) incorporated in a dialysate path as the dialysate passes the semipermeable membrane. In one embodiment, the sorbent is incorporated into the dialysate compartment or path within a hollow-fiber dialyzer. In another embodiment, the sorbent is incorporated into the dialysate path between two or more hollow-fiber dialyzers. The sorbent binds to non-urea uremic solutes and in particular to non-urea uremic solutes that bind to plasma proteins. In addition, the sorbent binds urea and inorganic ions much less effectively than non-urea uremic solutes and in particular non-urea uremic solutes that bind to plasma proteins.

### BRIEF DESCRIPTION OF THE DRAWINGS

FIG. 1 shows a standard hemodialysis 100 according to an exemplary embodiment of the invention. Blood 110 flows at a rate Q_{B} passed a semipermeable membrane 130 with size KoAₓ with dialysate 120 flowing at a rate Q_{D} in the opposite direction on the other side of the membrane. Uremic solutes (not shown) diffuse from the blood 110 into the dialysate 120 which goes down the drain.

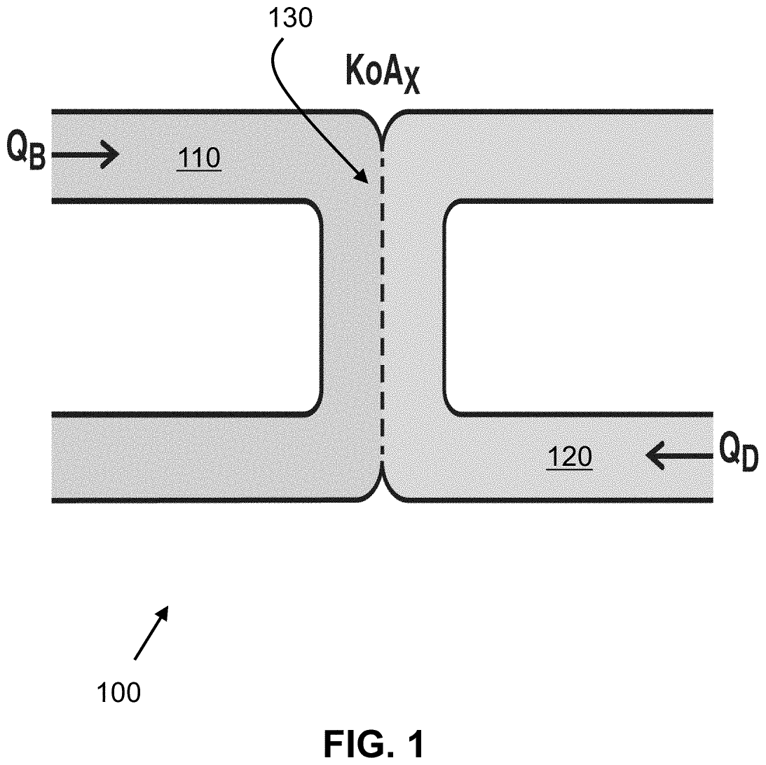

FIG. 2 shows a common hollow fiber dialyzer 200 for hemodialysis according to an exemplary embodiment of the invention.

FIG. 3 shows exemplary embodiment 300 of the invention where blood 310 flows through hollow fibers 312 which provide a semipermeable membrane 314 while dialysate 320 flows in the opposite direction. Dialysate 320 flows in respective dialysate compartments or path 322 around hollow fibers 312. Blood flow is indicated by down pointing arrows inside the hollow fibers 312. Dialysate flow is indicated by upward pointing arrows inside the dialysate compartments 322. The addition of a sorbent particles 330 the dialysate compartment or path 322 increases the clearance of solutes which bind to the sorbent by reducing the free concentration of these solutes in the dialysate toward zero. It is noted that the figure only depicts a few of the hollow fibers 312 and dialysate compartments/paths 322 in a hollow fiber hemodialyzer. The dimensions of these hollow fibers 312 through which the blood flows vary but their diameter is now often on the order of 200 um with a slightly lesser distance between the individual hollow fibers. Illustration to scale of sorbent particles 330 of any particular composition is not intended.

FIG. 4 shows an exemplary embodiment 400 of the invention where the clearance of protein-bound solutes could also be increased by inserting a sorbent cartridge 410 into the dialysis path between dialyzers of exiting structure.

FIG. 5 shows the potential ill effects of using low dialysate flow during home treatment. Plasma solute levels (vertical axis) in a dialysis patient as predicted by mathematical modeling are depicted through the 7 day (168 hour) weekly dialysis cycle (horizontal axis). Solute levels are reduced by each treatment and rise between treatments. The top panel depicts plasma levels of urea which is the standard dialysis index solute and the bottom panel depicts plasma levels of a solute which is largely bound to plasma proteins. The effect of low dialysate flow treatment, such as is commonly employed for home treatment, is depicted by lines 510 while the effect of standard in-center treatment is depicted by lines 520. Home treatment with low dialysate flow, here applied for 4 times per week for 5 hours, controls urea levels as well as standard in center treatment with higher dialysate flow, here applied 3 times per week for 3.33 hours. But low dialysate flow provides even less effective control of the protein-bound solute levels than standard treatment. The units on the vertical scale are arbitrary and intended to show only the relative plasma levels of urea and the bound solute with the two forms of treatment. The effect of the embodiments of the invention for low dialysate flow home treatment is that in the top panel the new line would be the same as line 510 as the invention would not lower urea levels. For the bottom panel, the effect of the embodiments of the invention is that the new line would be much lower than line 510 as the embodiments of the invention would lower levels of bound solutes so these levels would average as low or even lower than line 520.

### DETAILED DESCRIPTION

In a hemodialysis system, a sorbent is incorporated in the path of the dialysate as the dialysate passes by the dialysis membrane. In other words, this invention is not the procedure of adding a sorbent to the dialysate and pumping sorbent-containing dialysate past the dialysis membrane. Furthermore, this invention is also not a new membrane incorporating a sorbent. Instead embodiments of the invention are having a sorbent disposed along the path of the dialysate as the dialysate passes the dialysis membrane.

Such a design would improve the removal of numerous organic waste chemicals, denoted “uremic solutes”, while limiting the amount of dialysate required for hemodialysis treatment. At present, the adequacy of dialysis treatment is assessed by measuring removal of the single organic solute urea and the plasma levels of selected inorganic ions. Installation of a sorbent in the dialysate path as provided herein as the dialysate passes the dialysis membrane could increase removal of other uremic solutes relative to the removal of urea and thereby improve patients' health. Stated differently, embodiments of this invention would greatly increase the removal of protein-bound solutes as blood passes by the dialysis membrane, bringing the clearance of these solutes closer to that provided by the normal kidney.

Embodiments of this invention and the use of a sorbent differ from prior ways of using a sorbent as stated infra. In this invention, a sorbent is installed in the dialysate path as the dialysate flows on one side of the dialysis membrane while the blood flows on the other side of the dialysis membrane. This design and method provide at least the same increase in the clearance of bound solutes as the addition of a powdered sorbent to the dialysate, however, and this is key, without the modifications of the dialysate delivery system required for adding powdered sorbent to the dialysate. The sorbent design and use constitute a relatively simple technical means to improve cleansing of the blood at very modest cost.

Two designs are shown herein for installation of a sorbent in the dialysate path as the dialysate flows by the dialysis membrane. In both, the entire dialysate flow is passed over sorbent as the dialysate is pumped by one side of the dialysis membrane while the blood is pumped by the other side of the dialysis membrane. The characteristics of sorbents vary, but herein it is proposed to use sorbents which do not significantly bind urea or inorganic ions, but do bind many non-urea uremic solutes including particularly those that bind to plasma proteins. Examples of such sorbents useful for the embodiments in this invention include various forms of activated carbon, but other sorbents could also be used.

Design 1—Incorporation of a Sorbent into the Dialysate Compartment of a Hollow-Fiber Dialyzer

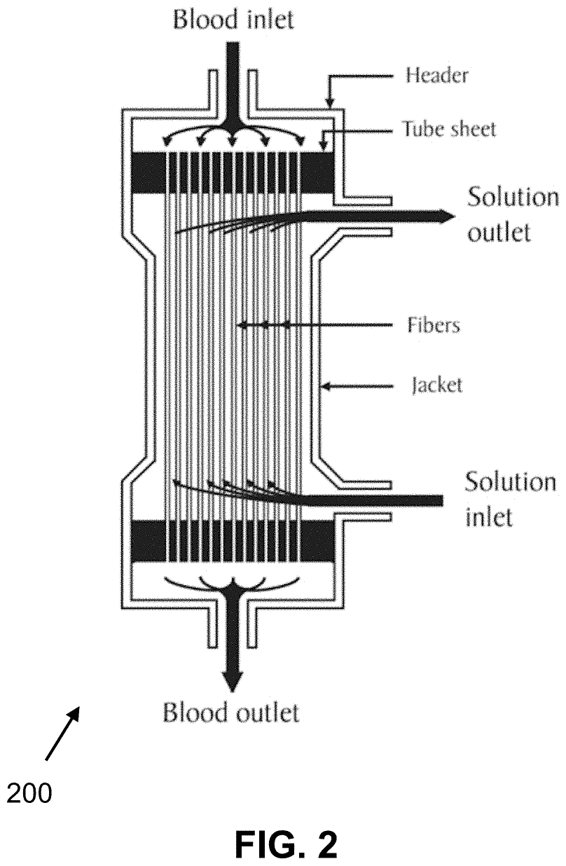

In modern dialyzers the blood passes through hollow fibers immersed in a stream of dialysate as shown in FIG. 2. These dialyzers are produced remarkably inexpensively and in the United States are usually discarded after a single use, so that annual usage exceeds 50 million units.

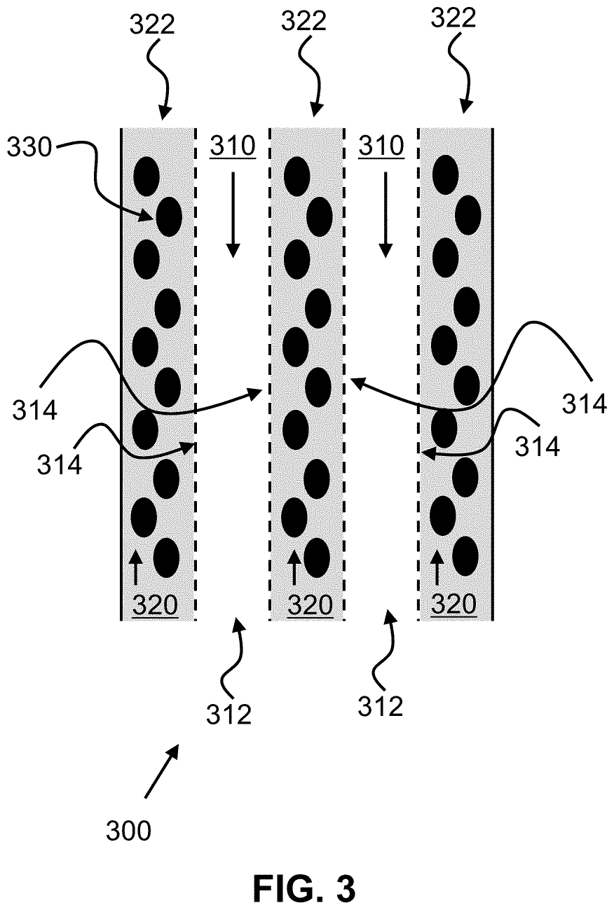

Incorporation of sorbents in the dialysate path would pose only a modest engineering and production challenge. In this embodiment, it is proposed to alter the construction of standard dialyzers by incorporating sorbent particles in the dialysate path as shown in FIG. 3.

FIG. 3 shows sorbent particles 330 interspaced between hollow fibers 312. Blood 310 flows through hollow fibers 312 of which the inner surface is semipermeable membrane 314 while dialysate 320 flows in the opposite direction on the other side of the semipermeable membrane 314. The addition of a sorbent particles 330 to the dialysate compartments or paths 322 increases the clearance of solutes which bind to the sorbent by reducing the free concentration of these solutes in the dialysate toward zero. Dimensions of hollow fibers now in use vary, but the diameter is often on the order of 200 μm with a slightly lesser distance between the fibers.

The inventor has found that powdered activated charcoal adheres spontaneously to the outside of polysulfone hollow fibers, but various additional means could be employed to fix sorbents in the dialysate path depending on fiber material and geometry and sorbent material and geometry. Such means include transforming the dialysate compartment into a carbon block.

Design 2—Incorporation of a sorbent into the dialysate path between two or more hollow-fiber dialyzers

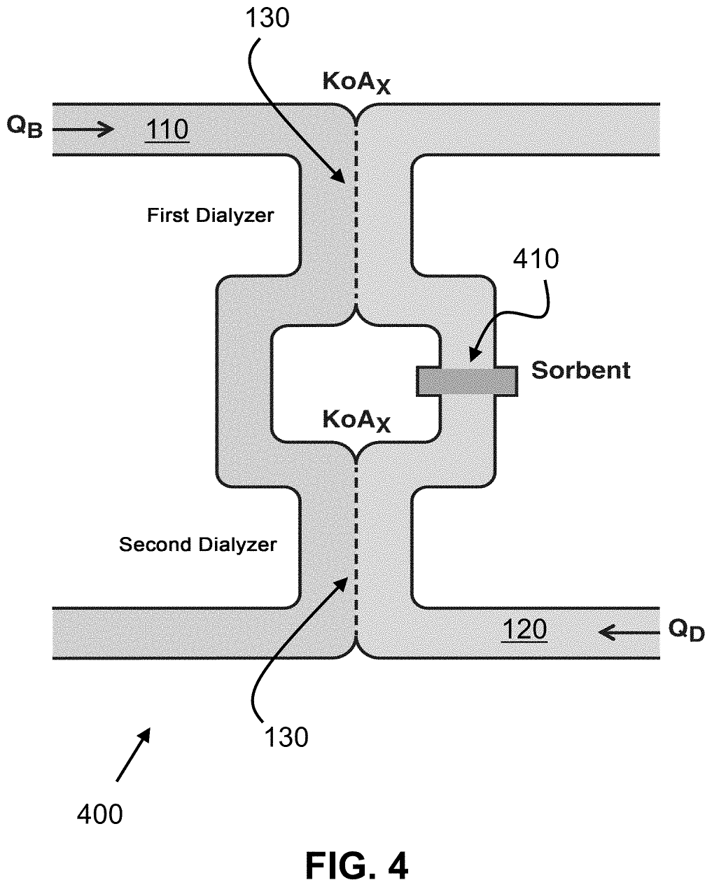

In an alternate embodiment, the same effect could be achieved as with design 1 by incorporating a sorbent into the dialysate path between two or more dialyzers as shown in FIG. 4. In this design, the incorporation of a sorbent into the dialysis path would only require production of a sorbent cartridge 410 and could be employed without significant alteration of current dialyzers or dialysis machines.

Home or Bedside Dialysis

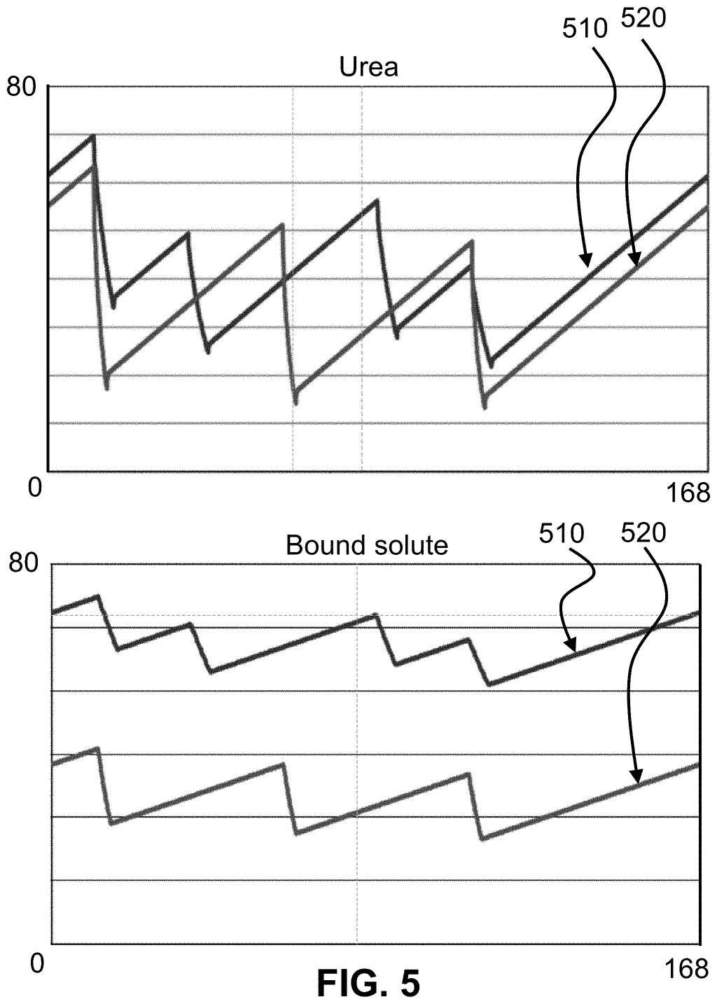

The incorporation of a sorbent into the dialysate path has value for home or bedside dialysis. Interest in dialysis machines for home use or institutional bedside use is growing rapidly. A major problem in the development of such systems has been the generation of dialysate. Dialysate for a home or bedside treatment must either be obtained by shipping bagged sterile fluid or local generation with special equipment. Both methods entail high costs and require space. One approach to the problem has been to minimize the ratio of dialysate flow to blood flow and thereby reduce the dialysate volumes employed each week. Such an approach combined with an increase in weekly treatment time and usually with an increase in weekly treatment frequency provides dialysis which meets current guidelines for urea removal and inorganic ion control. It may however provide inferior control of the levels of non-urea uremic solute as shown in FIG. 5. Embodiments of the present invention address these problems and make home dialysis more effective.

A low dialysate flow machine for home treatment has been available for 10 years, but has not come into widespread use. Many patients return from home treatment to standard treatment in dialysis centers. The inventor suspects that failure to control levels of non-urea solutes may contribute to the limited adoption of low dialysate flow treatment despite the advantages of flexible scheduling and the convenience of treatment in the home. Incorporation of a sorbent as provided by the embodiments described herein would be of particular value in this setting as it would increase the removal of protein-bound solutes without the use of higher dialysate flows.

What is claimed is:

1. A hemodialysis device comprising:
(a) a hemodialyzer designed for blood to flow past a semipermeable membrane and designed for a dialysate to flow in the opposite direction on the other side of the semipermeable membrane where uremic solutes are capable of diffusing from the blood into the dialysate passing the semipermeable membrane; and
(b) a sorbent incorporated in a dialysate path as the dialysate passes the semipermeable membrane.

2. The device as set forth in claim 1, wherein the sorbent is incorporated into the dialysate compartment or path within a hollow-fiber dialyzer.

3. The device as set forth in claim 1, wherein the sorbent is incorporated into the dialysate path between two or more hollow-fiber dialyzers.

4. The device as set forth in claim 1, wherein the sorbent binds non-urea uremic solutes or non-urea uremic solutes that bind to plasma proteins.

5. The device as set forth in claim 1, wherein the sorbent binds urea and inorganic ions much less effectively than non-urea uremic solutes or non-urea uremic solutes that bind to plasma proteins.

6. The device as set forth in claim 1, wherein the sorbent is an activated carbon.

7. A hemodialysis device comprising a hemodialyzer for blood to flow past a semipermeable membrane and designed for a dialysate to flow in the opposite direction on the other side of the semipermeable membrane where uremic solutes are capable of diffusing from the blood into the dialysate passing the semipermeable membrane, wherein the improvement comprises a sorbent incorporated in a dialysate path as the dialysate passes by the semipermeable membrane.

8. The device as set forth in claim 7, wherein the sorbent is incorporated into the dialysate compartment or path within a hollow-fiber dialyzer.

9. The device as set forth in claim 7, wherein the sorbent is incorporated into the dialysate path between two or more hollow-fiber dialyzers.

10. The device as set forth in claim 7, wherein the sorbent binds to non-urea uremic solutes or to non-urea uremic solutes that bind to plasma proteins.

11. The device as set forth in claim 7, wherein the sorbent binds urea and inorganic ions much less effectively than non-urea uremic solutes or non-urea uremic solutes that bind to plasma proteins.

12. The device as set forth in claim 7, wherein the sorbent is an activated carbon.

13. A hemodialysis method comprising:
(a) having a hemodialyzer designed for blood to flow past a semipermeable membrane and designed for a dialysate to flow in the opposite direction on the other side of the semipermeable membrane where uremic solutes are capable of diffusing from the blood into the dialysate passing the semipermeable membrane; and
(b) incorporating a sorbent in a dialysate path as the dialysate passes the semipermeable membrane.

14. The method as set forth in claim 13, wherein the sorbent is incorporated into the dialysate compartment or path within a hollow-fiber dialyzer.

15. The method as set forth in claim 13, wherein the sorbent is incorporated into the dialysate path between two or more hollow-fiber dialyzers.

16. The method as set forth in claim 13, wherein the sorbent binds to non-urea uremic solutes or to non-urea uremic solutes that bind to plasma proteins.

17. The method as set forth in claim 13, wherein the sorbent binds urea and inorganic ions much less effectively than non-urea uremic solutes or non-urea uremic solutes that bind to plasma proteins.

18. The method as set forth in claim 13, wherein the sorbent is an activated carbon.
